(19) United States
(12) Patent Application Publication
(10) Pub. No.: US 2025/0339301 A1
(43) Pub. Date: Nov. 6, 2025

(54) KNEE ORTHOSIS HAVING A TIBIAL CUFF LOCATOR

(71) Applicant: OSSKIN ORTHO INC., Montréal (CA)

(72) Inventors: Victor GRENIER, Montréal (CA); Farah OUALI, Montréal (CA); Pascale BRASSARD, Montréal (CA)

(21) Appl. No.: 19/195,011

(22) Filed: Apr. 30, 2025

Related U.S. Application Data
(60) Provisional application No. 63/641,175, filed on May 1, 2024.

(51) Int. Cl. A61F 5/01

(52) U.S. Cl. CPC A61F 5/0125; A61F 2005/0137; A61F 2005/0172

(57) ABSTRACT

A knee orthosis for a trans-tibial amputee having a leg prosthesis including a socket for receiving a stump, the knee orthosis has: a femoral cuff; a tibial cuff having an inner face facing the socket and an outer face, the tibial cuff defining a locating member; a hinge pivotally connecting the cuffs; and a tibial cuff locator including a body protruding from a base, the base having a prosthesis engaging surface adapted to be mounted on the leg prosthesis, the body of the tibial cuff locator extending outwardly from the base along a locator axis, the body being matingly engaged with the locating member of the tibial cuff, wherein translation of the tibial cuff relative to the tibial cuff locator in one or more directions transverse to the locator axis is mitigated with the tibial cuff locator matingly engaged with the locating member of the tibial cuff.

### CROSS-REFERENCE TO RELATED APPLICATIONS

The present application claims priority on U.S. Patent Application No. 63/641,175 filed on May 1, 2024, the entire content of which is incorporated herein by reference.

### TECHNICAL FIELD

The present disclosure relates generally to knee orthoses and, more particularly, to knee orthoses configured to be securely located on the wearer's leg.

### BACKGROUND

For some individuals, muscles, ligaments and other soft tissue of a knee need to be stabilized. This may be the result of injuries, medical procedures, and so on. A knee orthosis may be used for this purpose. Knee orthoses may also be used in specific cases, for example for stabilizing the knee of an amputee or a person having lower limb differences.

For example, trans-tibial amputees have their knee joint preserved but often have pathological biomechanics as the residual limb and prosthesis do not offer the same stability/control as the native limb. This may result in knee ligament deficiencies, which require stabilization. While knee orthoses may be used to provide such stabilization, existing knee orthoses, such as those which use a polycentric design, are not ideally suited to bracing the knees of trans-tibial amputees or wearers of other prosthetic devices. Movements between the knee orthosis and the leg, or between the knee orthosis and a lower leg prosthesis, is undesirable as it can result in unwanted forces and/or migration between the wearer's leg and the knee orthosis. In the particular case of trans-tibial amputees, the limited amount of soft tissue available to compensate for the relative position of the knee orthosis and the limb also causes greater challenges in ensuring that the knee orthosis accurately tracks the natural motion of the leg and knee. Improvements are therefore sought.

### SUMMARY

In one aspect, there is provided a knee orthosis for a trans-tibial amputee having a leg prosthesis including a socket for receiving a stump, the knee orthosis comprising: a femoral cuff configured for engaging an upper leg portion of the trans-tibial amputee; a tibial cuff configured for engaging the socket of the leg prosthesis, the tibial cuff having an inner face facing the socket and an outer face opposed to the inner face, the tibial cuff defining a locating member; a hinge pivotally connecting the femoral cuff to the tibial cuff; and a tibial cuff locator including a body protruding from a base, the base of the tibial cuff locator having a prosthesis engaging surface adapted to be mounted on the leg prosthesis, the body of the tibial cuff locator extending outwardly from the base along a locator axis, the body of the tibial cuff locator being matingly engaged with the locating member of the tibial cuff, wherein translation of the tibial cuff relative to the tibial cuff locator in one or more directions transverse to the locator axis is mitigated with the tibial cuff locator matingly engaged with the locating member of the tibial cuff.

The knee orthosis defined above and described herein includes, in certain embodiments, one or more of the following features, in whole or in part, and in any combination.

In some embodiments, the locator axis is transverse to the socket engaging surface of the base of the tibial cuff locator.

In some embodiments, the tibial cuff locator is adapted to be mounted on the leg prosthesis such that the tibial cuff is engageable to the socket in a single position of the tibial cuff relative to the socket.

In some embodiments, the tibial cuff locator and the locating member of the tibial cuff have at least partially complementary geometries.

In some embodiments, the body of the tibial cuff locator has a substantially circular cross-section transverse to the locator axis.

In some embodiments, the tibial cuff locator defines a central hole extending along the locator axis and through the tibial cuff locator, the central hole sized to receive a marking device for imprinting a mark on the socket with the marking device received through the central hole.

In some embodiments, the tibial cuff locator is configured to remain secured to the leg prosthesis to ensure that a position of the knee orthosis relative to the leg prosthesis is constant between successive installations of the knee orthosis.

In some embodiments, the tibial cuff locator and the tibial cuff define, when mated together, a load path extending from the tibial cuff to the socket through the tibial cuff locator.

In some embodiments, the locating member includes a recess defined by the tibial cuff, the body received within the recess, a shape of the body corresponding to a shape of the recess.

In some embodiments, the base has a length and a width transverse to the length, the length being parallel to a socket central axis of the socket, the length greater than the width.

In some embodiments, the recess is an aperture extending fully through the tibial cuff.

In some embodiments, the tibial cuff locator is located on an antero-lateral side of the knee orthosis.

In some embodiments, the tibial cuff locator and the locating member of the tibial cuff are matingly engaged to prevent relative rotation between the tibial cuff locator and the tibial cuff about the locator axis.

In some embodiments, a body of the locator has a non-circular cross-sectional area taken on a plane normal to the locator axis.

In some embodiments, a locking member removably fastens the knee orthosis and the tibial cuff locator together.

In another aspect, there is provided a knee orthosis for a trans-tibial amputee having a leg prosthesis including a socket for receiving a stump, the knee orthosis comprising: a femoral cuff configured for engaging an upper leg portion of the trans-tibial amputee; a tibial cuff configured for engaging the socket of the leg prosthesis, the tibial cuff having an inner face facing the socket and an outer face opposed to the inner face, the tibial cuff defining a locating member, the locating member defining an abutment face configured to abut a tibial cuff locator, the abutment face being transversal to the outer face of the tibial cuff, the abutment face and the tibial cuff locator adapted to abut one another to limit relative sliding movement between the tibial cuff and the leg prosthesis in one or more directions; and a hinge pivotally connecting the femoral cuff to the tibial cuff.

The knee orthosis defined above and described herein includes, in certain embodiments, one or more of the following features, in whole or in part, and in any combination.

In some embodiments, the one or more directions includes a direction that is transverse to the tibial cuff locator and/or a direction that is parallel to a longitudinal axis of a leg of a wearer.

In some embodiments, the tibial cuff locator is circular.

In some embodiments, the locating member is a recess defined by the tibial cuff, the tibial cuff locator received in the recess.

In some embodiments, a body of the locator has a non-circular cross-sectional area taken on a plane normal to a locator axis of the body of the locator.

In yet another embodiment there is provided a knee orthosis comprising: a femoral cuff pivotally connected to a tibial cuff, the femoral cuff configured for engaging an upper leg portion of a wearer of the knee orthosis and the tibial cuff configured for engaging a lower leg portion of the wearer, the tibial cuff having an inner face and an outer face opposed to the inner face, the tibial cuff defining a locating member; and a tibial cuff locator including a body protruding from a base, the base of the tibial cuff locator adapted to be mounted on the lower leg portion, the body of the tibial cuff locator extending outwardly from the base along a locator axis, the body of the tibial cuff locator being matingly engaged with the locating member of the tibial cuff, wherein translation of the tibial cuff relative to the tibial cuff locator in one or more directions transverse to the locator axis is mitigated with the tibial cuff locator matingly engaged with the locating member of the tibial cuff.

The knee orthosis defined above and described herein may also include, in certain embodiments, any of the above-noted features, in whole or in part, and in any combination.

### BRIEF DESCRIPTION OF THE DRAWINGS

Reference is now made to the accompanying figures in which:

FIG. 1 is a three-dimensional view of a knee orthosis for a trans-tibial amputee in accordance with one implementation of the present technology, the knee orthosis being worn by the trans-tibial amputee having a leg prosthesis;

FIG. 2 is a three-dimensional view of a socket of the leg prosthesis of FIG. 1;

FIG. 3 is a front three-dimensional view of the knee orthosis of FIG. 1;

FIG. 4 is another three-dimensional front view of the knee orthosis worn by the trans-tibial amputee and illustrating a method of installation thereof;

FIG. 5 is a three-dimensional exploded view illustrating a tibial cuff locator and a locating member of the knee orthosis being lockable to one another via a locking member;

FIGS. 6A and 6B are front and side views, respectively, of a tibial cuff locator in accordance with another embodiment;

FIG. 7 is a three-dimensional view of a knee orthosis in accordance with another embodiment;

FIG. 8 is a front view of a tibial cuff locator to be used with the knee orthosis of FIG. 7;

FIG. 9 is a three-dimensional view of a portion of a knee orthosis in accordance with yet another embodiment;

FIG. 10 is a three-dimensional view of a tibial cuff locator to be used with the knee orthosis of FIG. 9; and

FIG. 11 is a schematic front view of yet another embodiment of a knee orthosis and tibial cuff locator.

### DETAILED DESCRIPTION

Knee orthoses are externally applied devices used to influence the structural and functional characteristics of the neuromuscular and skeletal systems. A knee orthosis is a brace that extends above and below the knee joint and is generally worn to support or align the knee during flexion thereof. In the case of conditions affecting the ligaments or cartilage of the knee, a knee orthosis can provide stabilization to the knee by replacing or assisting the function of these injured or damaged ligaments or cartilage of the knee.

Stabilizing a transtibial amputee's knee using a knee orthosis (which may also be referred to herein as a “knee brace” or simply “brace”) offers several advantages, as it is easily removable and can be worn whenever the patient or clinical feels its necessary (e.g., for higher intensity activities). The residual limb (or stump) and socket can have varying shapes and sizes from one patient to another. Furthermore, there is no soft tissue on the tibia to compensate for discrepancies in socket vs. knee orthosis surface fit. Therefore, a custom, scan-based solution is preferred to a prefab knee orthosis with a typically cylindrical rigid cuff.

It is desirable for the knee orthosis to properly track a natural motion of the leg and knee (with a plurality of DOFs, screw-home mechanism, etc.) during flexion. If the leg and knee orthosis do not move together, there may be undesirable forces and slippage (i.e., migration) between the two.

This effect is worsened with trans-tibial amputees, as less soft tissue (skin, muscles, fat) exists to compensate for the relative position of the knee orthosis relative to the limb. As such, conventional polycentric knee orthoses are not well suited to bracing these patients. Put differently, gravity combined with repeated movements of the leg of the transtibial amputee may cause the knee orthosis to become misaligned relative to a leg prosthesis of the amputee. The relative migration of the knee orthosis may prevent the latter from properly assisting a knee flexion of the amputee. The present knee orthosis seeks to address at least some of these issues.

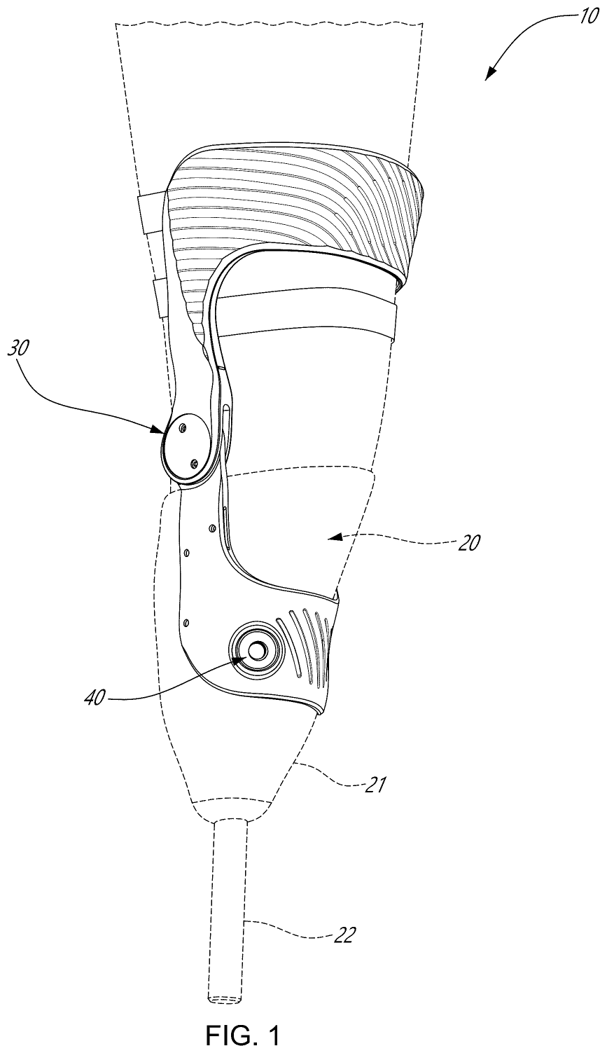

Referring to FIG. 1, an assembly 10 of a leg prosthesis 20 for a trans-tibial amputee and a knee orthosis 30 that may at least partially alleviate the afore-mentioned drawbacks is shown. The leg prosthesis is shown at 20 and the knee orthosis is shown at 30. The leg prosthesis 20 includes a socket 21 for receiving a stump of the trans-tibial amputee and a leg member 22 that extends from the socket 21 towards a foot (not shown). The knee orthosis 30 is used to help stabilizing the socket 21 of the leg prosthesis 20. The knee orthosis 30 may be custom fitted using a 3D-scan of the leg and prosthesis 20.

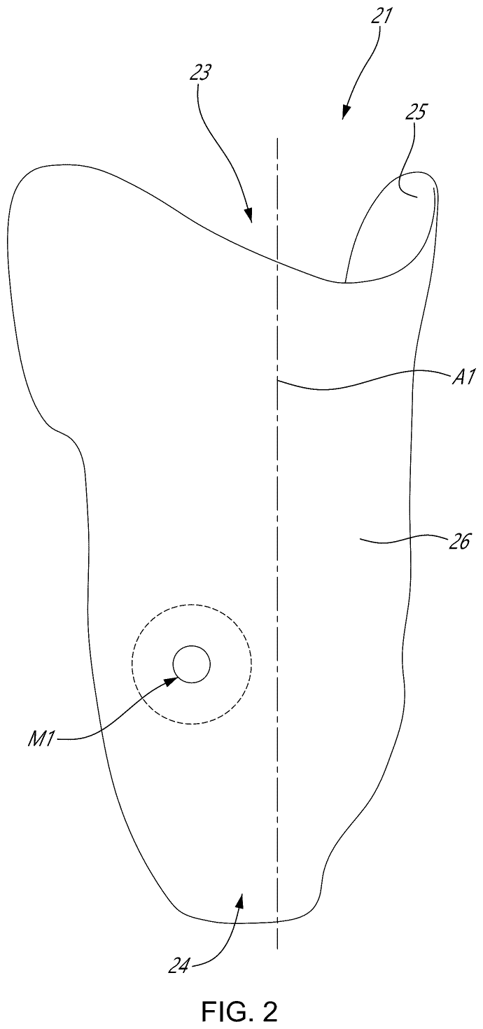

Referring to FIG. 2, the socket 21 extends along a socket central axis A1 from an open end 23 to a closed end 24. The wearer inserts an end of his or her leg into the socket 21 via the open end 23 until the end abuts the closed end 24. The socket central axis A1 is therefore substantially parallel to a tibial bone of the wearer. In the context of the present disclosure, the expression “substantially” as in “substantially parallel” implies slight deviations from parallel due to an anatomy of the wearer, movements, and so on. The shape of the socket 21 is selected to substantially match that of the stump of the wearer. The socket 21 has an inner surface 25 facing an internal volume that receives the stump and an outer surface 26 opposed to the inner surface 25.

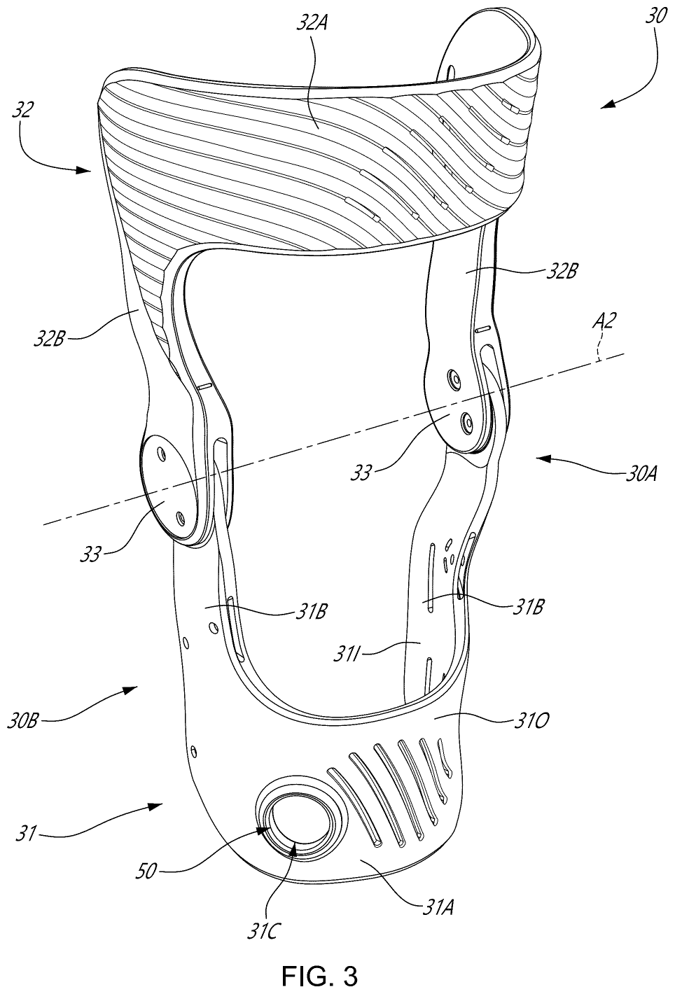

Referring to FIG. 3, the knee orthosis 30 is shown in greater detail. The knee orthosis 30 includes a tibial cuff 31 and a femoral cuff 32. The tibial cuff 31 may be secured, such as with straps, to the socket 21, and extends partially around the socket 21 and abuts the outer surface 26 of the socket 21. The tibial cuff 31 is configured for engaging the socket 21 of the leg prosthesis 20. Put differently, the tibial cuff 31 has a curved shape configured to mate with the socket 21. The femoral cuff 32 extends partially around a thigh of the wearer and is securable to the thigh via any suitable means, such as straps. In other words, the femoral cuff 32 is configured for engaging an upper leg portion of the trans-tibial amputee. The femoral cuff 32 may have a curved shape configured to mate with a thigh of the wearer. The tibial cuff 31 is hingedly or pivotally connected to the femoral cuff 32 via a hinge, which includes two hinges 33, which may also be referred to as pivots. The two hinges 33 allow rotation of the tibial cuff 31 relative to the femoral cuff 32 about a pivot axis A2, which may be substantially coaxial with a pivot axis of a knee of the wearer.

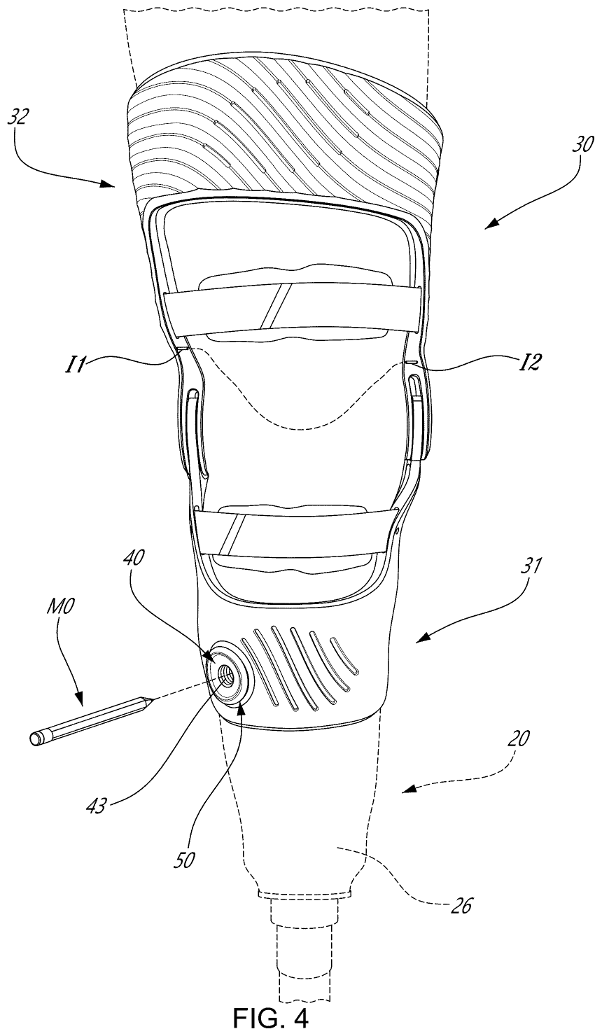

As shown in FIG. 4, the femoral cuff 32 has two indicator lines 11, 12 each disposed on a respective one of two sides of the knee orthosis 30. These lines 11, 12 are meant to facilitate an installation of the knee orthosis 30 on the socket 21 by aligning these lines 11, 12 with a top of the socket 21. These lines may be omitted in some embodiments.

Referring back to FIG. 3, as illustrated, the knee orthosis 30 has a medial side 30A and a lateral side 30B. Both of the tibial cuff 31 and the femoral cuff 32 extend from the medial side 30A to the lateral side 30B of the knee orthosis 30. Put differently, the medial side 30A of the knee orthosis 30 and one of the two hinges 33 is located adjacent a medial side of a knee of the wearer and the lateral side 30B of the knee orthosis 30 and the other of the two hinges 33 is located adjacent a lateral side of the knee. The knee orthosis 30, and both of the tibial cuff 31 and the femoral cuff 32, define a curved shape configured to substantially conform to a front section of the leg of the wearer. In an alternate embodiment, at least one of the tibial and femoral cuffs 31, 32 may be shaped to mate with a posterior side of the leg of the wearer. For example, the tibial cuff 31 may be located on a posterior side of the leg and thus be shaped to conform to the posterior side of the leg of the wearer.

More specifically, the tibial cuff 31 has a tibial transverse member 31A wrapping around a portion of the socket 21 and around the socket central axis A1 and has two tibial longitudinal members 31B protruding from the tibial transverse member 31A away from the closed end 24 (FIG. 2) of the socket 21 and substantially along the socket central axis A1. Each of the two tibial longitudinal members 31B are disposed on a respective one of the medial side 30A and the lateral side 30B of the knee orthosis 30. Similarly, the femoral cuff 32 has a femoral transverse member 32A wrapping around a portion of the thigh of the wearer and around a longitudinal axis of a femoral bone of the wearer, and has two femoral longitudinal members 32B protruding from the femoral transverse member 32A towards the knee of the wearer and substantially along the longitudinal axis of the femoral bone. Each of the two tibial longitudinal members 31B is pivotably connected to a respective one of the two femoral longitudinal members 32B at a respective one of the two hinges 33.

Referring now to FIG. 4, in the embodiment shown, a tibial cuff locator 40 is used to position the knee orthosis 30 relative to the socket 21. The tibial cuff locator 40 (which may also be simply referred to as the “locator” 40) may further be used to transfer a load from the socket 21 to the tibial cuff 31. Hence, a load path may extend from the tibial cuff 31 to the socket 21. The load path extends through the tibial cuff locator 40. As shown in FIG. 4, the tibial cuff locator 40 is secured to the outer surface 26 of the socket 21. In some embodiments, the tibial cuff locator 40 is located on an antero-lateral side of the knee orthosis 30. Other locations are contemplated. The tibial cuff locator 40 may be integrated to or secured to any part of the leg prosthesis 20. In other words, the socket 21, or other parts of the leg prosthesis, may define the locator 40. The locator 40 and the socket 21 may be two parts of a single monolithic body. The tibial cuff locator 40 cuff may be positioned anteriorly, but it could be posterior-, medial- or laterally positioned. It is however more convenient to have an antero-lateral position, as the anterior cuff may be easily placed or folded over the locator 40 during fitting. Also, the locator 40 may be positioned closer to one of the tibial longitudinal members 31B, which may define a thickest part of the tibial cuff 31. This may offer more mechanical rigidity than if the locator 40 were centered anteriorly on the tibial cuff 31. The disclosed position of the locator 40 may minimize interference, rubbing, or hitting the other limb of the wearer.

As described herein, the tibial cuff locator 40 is secured to the leg prosthesis 20, namely to the socket 21 of the leg prosthesis. It will however be appreciated that the expression “leg prosthesis” should encompass any device worn by a patient (e.g., trans-tibial amputee, or any patient or user having a rigid support on his or her leg), such as, for instance, an AFO or another orthotic or lower limb rigid support. In certain other embodiments, the knee orthosis 30 may be used in conjunction with other types of lower limb rigid supports, such as knee-ankle-foot orthoses (KAFO), or other types of orthotic, prosthetic, sporting (e.g., ski boots) or other structures. Accordingly, it is to be understood that knee orthosis as described herein may be used either by trans-tibial amputees, or by other (e.g., non-amputee) users. In certain embodiments, the base 41 of the tibial cuff locator 40 may be adapted to be mounted directly on a lower leg portion of a wearer. For example, the base 41 may be affixed, such as with glue, straps, or other suitable fastening means, directly on the skin of the wearer (who may or may not be an amputee).

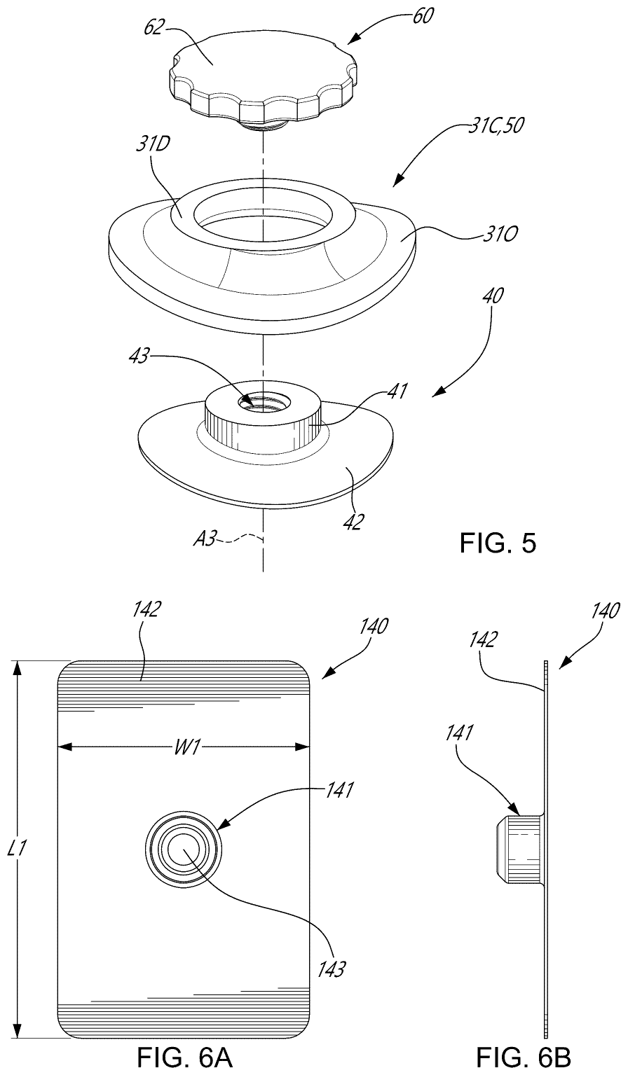

Referring to FIG. 5, the tibial cuff locator 40 includes a body 41 protruding from a base 42 along a body axis, also referred to as a locator axis A3. The base 42 defines a socket engaging surface, which may also be referred to as a prosthesis engaging surface, adapted to be mounted on the socket 21 of the leg prosthesis 20, or to any other part of the prosthesis. The locator axis A3 is transverse to the socket engaging surface of the base 42 of the tibial cuff locator 40. The body 41 is rigid and serves to take a load from the tibial cuff 31, which may be caused by slippage and gravity, and transfers it through the base 42 and the socket 21. The base 42 is securable to the outer surface 26 of the socket 21, such as via an adhesive or any other suitable fastening means (e.g., mechanical fasteners, keyway engagement, etc.). The base 42 may be thin and pliable to wrap on any possible socket surface shape. In this configuration, the base 42 has an annular shape and it may be suitably curved to be able to mate with an external surface of the socket 21. However, as will be discussed below, the shape of the base 42 may vary in some other embodiments. In some embodiments, the socket 21 and the tibial cuff locator 40 may be two parts of a single monolithic body. Put differently, the socket 21 may define the tibial cuff locator 40. The body 41 extends from the base 42 to a distal end, which may be chamfered to facilitate its engagement to the tibial cuff 31 as will be discussed below.

As shown in FIG. 5, a cross-sectional area of the body 41 taken on a plane normal to the body axis A3 is substantially circular. Herein, “substantially” imply slight deviations from perfectly circular due to a manufacturing tolerances for instance. This may facilitate an engagement of the tibial cuff locator 40 to the knee orthosis 30 as will be discussed below. The circular shape may be well suited to accommodate slight misalignment during the placement of the tibial cuff locator 40 and may allow obtaining proper relative angular position between the knee orthosis 30 and the socket 21. The body 41 has a sufficient thickness or height along the body axis A3 to ensure efficient suspension. Moreover, the height of the body 41 may prevent tilting of the socket 21 relative to the knee orthosis 30. Also, a height of the body 41 taken from the base 42 to the distal end along the body axis A3 may be sufficient to prevent slippage of the body 41 relative to the tibial cuff 31. The height is selected to prevent the tibial cuff locator 40 from getting disengaged from the tibial cuff 31. Put differently, the height may be selected to allow the body 41 to exert a moment on the knee orthosis 30 about an axis being transverse to the body axis A3.

Referring back to FIG. 3, the body 41 of the tibial cuff locator 40 is engageable to a locating member 50 of the tibial cuff 31. In the embodiment shown, the locating member 50 is a recess 31C defined by the tibial transverse member 31A of the tibial cuff 31 and having a shape corresponding to that of the body 41 of the tibial cuff locator 40. In other words, the tibial cuff locator 40 and the locating member 50 of the tibial cuff have at least partially complementary geometries. Complementary geometries may include, for instance, a hole and a pin, a dog and a slot, a tongue and a groove and so on. Any complementary geometries that include two members engageable to one another may be used. The tibial cuff 31 has an inner face 311 facing the socket 21 and an outer face 310 opposed to the inner face 311. The recess 31C extends from the inner face 311 towards the outer face 310. The recess 31C may extend all the way through from the inner face 311 to the outer face 310. Put differently, the recess 31C is, in the illustrated embodiment, an aperture extending through the tibial transverse member 31A. It may however be a cavity, dimple and so on defined by the tibial transverse member 31A. The shape of the recess 31C or aperture matches a profile of the locator 40.

The knee orthosis 30 is positioned over the locator 40 by inserting it in its dedicated hole, providing a stable/repeatable cuff position. Put differently, the tibial cuff locator 40 may cause the tibial cuff 31 to be engaged to the socket 21 via the tibial cuff locator 40 in a single position of the tibial cuff 31 relative to the socket 21. In other words, the tibial cuff 31 is engaged to the tibial cuff locator 40 in a single position of the tibial cuff 31 relative to the socket 21. In the disclosed embodiment, the tibial cuff locator 40 is removably matingly engaged within the recess 31C of the tibial cuff 31 to prevent relative movements between the tibial cuff locator 40 and the tibial cuff 31 in a direction parallel to the inner face of the tibial cuff 31. In other words, the tibial cuff locator 40 and the recess 31C of the tibial cuff 31 are matingly engaged to prevent relative movement between the tibial cuff locator 40 and the tibial cuff 31 along the inner face of the tibial cuff 31. Stated differently, the tibial cuff locator 40 protrudes along the locator axis A3, which is transverse to the inner face 311 of the tibial cuff 31. By this engagement, relative translation of the tibial cuff 31 relative to the socket 21 along directions being transverse to the locator axis A3 are prevented by the tibial cuff locator 40 engaging the locating member 50 of the tibial cuff 31. In other words, translation of the tibial cuff 31 relative to the tibial cuff locator 40 in one or more directions transverse to the locator axis A3 is mitigated with the tibial cuff locator 40 matingly engaged with the locating member 50 of the tibial cuff 31. It will be appreciated that small movements are possible due to small difference between an inner diameter of the recess 31C and an outer diameter of the tibial cuff locator 40. This small difference may be caused by manufacturing tolerances and may be desired to avoid a tight fit between these two components. However, in some configurations, a tight fit or frictional engagement between the locator and the cuff may be present. The tibial cuff locator 40 is engaged to the recess 31C to prevent movement between these two components in a longitudinal direction and/or a transversal direction. The longitudinal direction has a component in a plane parallel to a sagittal plane of the wearer. The transversal direction has a component in a plane parallel to a coronal or frontal plane of the wearer.

The tibial cuff locator 40 may facilitate the repeatable positioning of the tibial cuff on the leg prosthesis 20. In other words, the tibial cuff locator 40 is used as a positioning feature to repeatedly install the knee orthosis 30 on the leg prosthesis 20. Thus, the wearer may not be required to adjust the position of the knee orthosis 30 relative to the leg prosthesis 20 each time he or she wants to use the knee orthosis 30. As the leg prosthesis' socket 21 is typically molded to the patient's stump, its position relative to the limb is repeatable when installed. As the knee orthosis 30 is repeatably positioned relative to the leg prosthesis 20 thanks to the tibial cuff locator 40, is it therefore also repeatably and adequately aligned to the limb during installation. Moreover, once the knee orthosis 30 has been installed on the leg prosthesis 20, the tibial cuff locator 40 contributes in preventing migration (any unwanted movement) of the knee orthosis 30 relative to the leg prosthesis 20. In the context of the present disclosure, the expression “migration” may include a translation of the knee orthosis 30 relative to the leg prosthesis 20.

The disclosed tibial cuff locator 40 may cooperate with the knee orthosis 30 to minimize migration of the socket 21 relative to the leg of the wearer, and to improve repeatability of knee orthosis placement on the knee. The locator 40 makes use of the repeatable positioning of the socket 21 on the stump to fix the knee orthosis 30 on the socket 21.

Referring to FIG. 4, a method of installing the tibial cuff locator 40 is now described. As shown, the tibial cuff locator 40 defines a hole 43, which may be coaxial with the locator axis A3. The hole 43 is depicted as being circular, but other shapes are contemplated. The hole 43 is sized to receive a marker M0, such as a pencil or other marking device, used to create a mark M1 (FIG. 2) at a location of where the tibial cuff locator 40 is to be secured on the socket 21, or other parts of the leg prosthesis. To secure the tibial cuff locator 40 on the socket 21, the tibial cuff locator 40 is engaged to the locating member 50. In this embodiment, this implies inserting the body 41 into the recess 31C. At which point, the knee orthosis 30 is installed to the wearer and the tibial cuff 31 is abutted against the socket 21 thereby abutting the base 42 of the tibial cuff locator 40 to the socket 21. The marker M0 is inserted in the hole 43 of the tibial cuff locator 40 to mark out a position of the tibial cuff locator 40 on the socket 21. Then, the knee orthosis 30 is removed and the tibial cuff locator 40 may be secured to the socket 21 by aligning the hole 43 with the mark that has been created on the socket 21. The base 42 may be secured to the socket 21 or to another portion of the prosthesis via an adhesive, mechanical fasteners (e.g., rivets, bolts, etc), or any other suitable fastening means.

In other words, the wearer is asked to extend his or her leg and the knee orthosis 30 is positioned with the tibial cuff locator 40 pre-inserted in the recess 31C. The knee orthosis 30 is strapped on the leg (both tibial cuff 31 and femoral cuff 32) and the position of the locator 40 is marked on the socket 21 using a white pen (or any suitable color) through the hole 43 of the locator 40. The brace is then removed from the patient's leg and the locator 40 separated from the knee orthosis 30. A film covering an adhesive on the base 42 of the locator 40 is removed. This step may differ should another fastening mean is used. The locator 40 is taped on the socket making sure to match the locator's hole 43 on the marking M1 priorly made on the socket 21 and placing the base 42 of the locator 40 substantially parallel to the socket central axis A1. Pressure may be applied to the base 42 of the locator 40 to ensure proper adhesion. The wearer may be asked to wait until the adhesive is properly cured.

Referring now to FIG. 5, as illustrated, the tibial cuff locator 40 has its body 41 received through the recess 31C defined by the knee orthosis 30. As shown, the knee orthosis defines a lip 31D that surrounds the recess 31C. The lip 31D protrudes away from the outer face 310 of the knee orthosis 30 and away from the trans-tibial amputee. In some embodiments, it may be desired to lock the knee orthosis 30 to the tibial cuff locator 40, and thus to the leg prosthesis to which the tibial cuff locator is mounted. To do so, a locking member 60 is provided to removably fasten (i.e., to selectively lock and unlock, when desired) the knee orthosis 30 and the tibial cuff locator 40 together. The locking member 60 includes, in the depicted embodiment, a knob including a threaded shank that is inserted through the recess 31C and threadedly engaged with corresponding threads defined by the hole 43 of the tibial cuff locator 40. The locking member 60 may be tightened until its head 62 abuts against the lip 31D. A height of the lip 31D is thus selected to ensure that the locking member 60 does not exert too much force that may cause detaching of the tibial cuff locator 40 from the socket 21. Any other suitable locking member may be used, such as a lock button, a keyway engagement, a dog and slot, a bayonet lock, a hook, and so on. Referring now to FIGS. 6A-6B, another embodiment of the tibial cuff locator is shown at 140. As illustrated, a base 142 of the tibial cuff locator 140 has a substantially rectangular shape, with a length L1 being greater than a width W1. The length L1 is parallel to the socket central axis A1, or substantially parallel to a leg of the wearer. Having the length L1 greater than the width W1 may provide a better transfer of force in the direction in which the wearer is going to apply force on the knee orthosis 30. A body 141 of the tibial cuff locator 140 may be centered on the base 142. The body 141 defines the central hole 143 to allow the marking of the location on the socket 21 as already explained above.

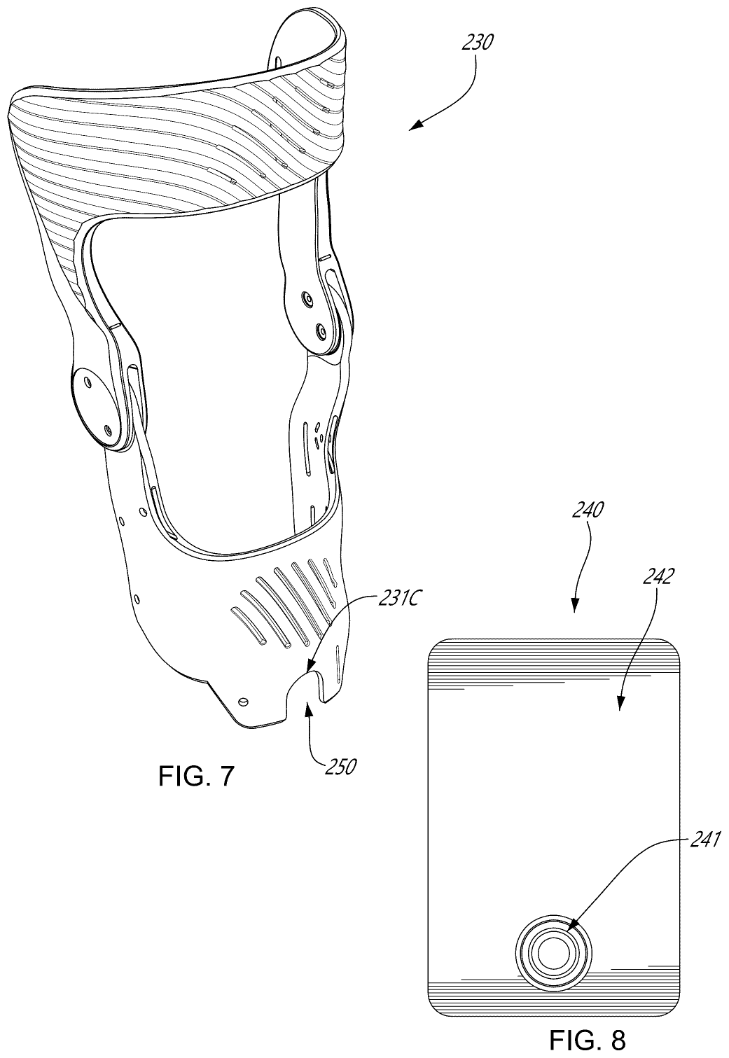

Referring now to FIGS. 7-8, another embodiment of a knee orthosis and of a tibial cuff locator are shown at 230 and 240, respectively. For the sake of conciseness, only features differing from the knee orthosis 30 described above and of the tibial cuff locator 40, 140 described above will be described below. As illustrated, the knee orthosis 230 includes a locating member 250. In this embodiment, the locating member 250 is a notch 231C defined at a bottom end of the tibial cuff. The tibial cuff locator 240 is similar to the tibial cuff locator 140 described above with reference to FIGS. 6A-6B. However, since the locating member 250 is located lower on the tibial cuff, a body 241 of the tibial cuff locator 240 is displaced longitudinally closer to a bottom end of a base 242 of the locator. The base 242 may have a length greater than the width as described above with reference to FIGS. 6A-6B. Displacing the body 241 lower may ensure that the base 242 has sufficient area to be adhered to the socket 21. The notch 231C may be a groove, outline or any suitable features engageable by the locator 240.

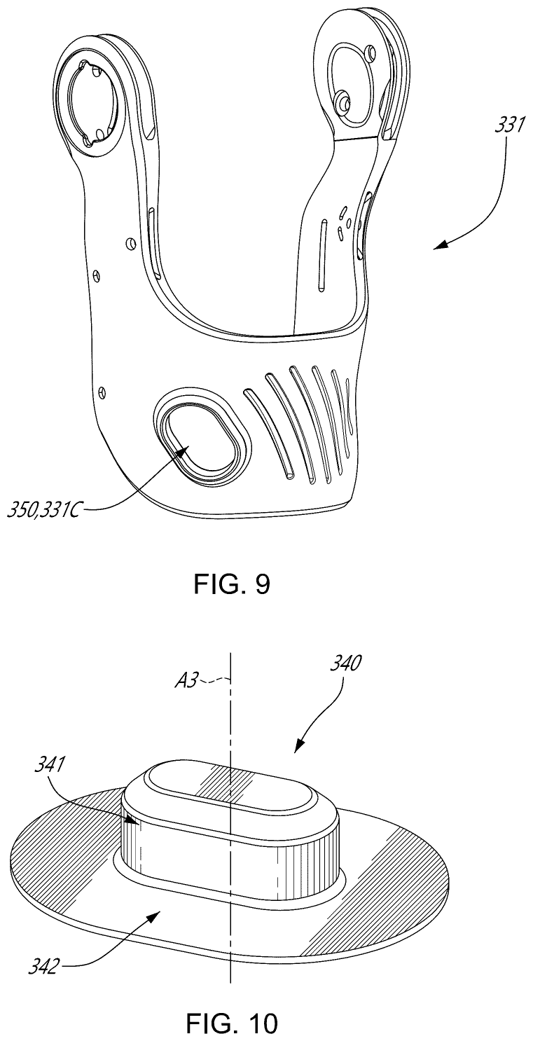

Referring to FIGS. 9-10, another embodiment of a knee orthosis and of a tibial cuff locator are shown respectively at 330 and 340. In this embodiment, a locating member 350 of a tibial cuff 331 corresponds to a recess 331C defined through the tibial cuff 331. The recess 331C has a non-circular shape (e.g., oval, ellipse, rectangular, etc.). Correspondingly, a body 341 of the tibial cuff locator 340 also has a non-circular shape that is sized to engage the recess 331C of the tibial cuff 331. More specifically, the body 341 has a length and a width being transverse to one another and transverse to the body axis A3. The length is greater than the width. A base 342 may extend annularly around the body 341 and around the body axis A3. In an alternate embodiment, the base 342 need not extend fully annularly around the body axis A3. For instance, the base 342 may have an epitrochoid shape (e.g., oval shape with concave sides) or other shapes such as an oblong or dog-bone shape, to facilitate its adhesion and conformance on the socket 21. The base may alternately have a triangular shape, or may be non-uniform about a portion or an entirely of the perimeter of the body 41. Other suitable shapes are contemplated, and the clinician can cut the shape of the base as needed. The base 342 may be secured to the socket 21 or to another portion of the prosthesis via an adhesive, mechanical fasteners (e.g., rivets, bolts, etc), or any other suitable fastening means. The base 342 may be thin and pliable to wrap on any possible socket surface shape. The body 341 is rigid and may be centered on the base 342. The body 341 serves to take the load from the tibial cuff 331, which may be caused by slippage and gravity, and transfer it through the base 342 and the socket 21.

The profile of the body 341 may vary, but, in this configuration, it has a slightly elongated profile, with the longer dimension aligned approximately along the axial axis of the limb. As the knee orthosis 30 slips down the leg and socket 21, the applied shear force is axial. The elongated profile may then provide a mechanical advantage, making it harder for the locator to ‘tip’ and fall off.

In the embodiment shown, the body 341 is positioned on the socket 21 with the length oriented substantially parallel to the socket central axis A1. In other words, a projection of the length of the body 341 on the socket central axis A1 is greater than a projection of the width of the body 341 on an axis being perpendicular to the socket central axis A1. Stated differently, the body 341 is aligned such that the length is more aligned with the socket central axis A1 than the width W1. Moreover, as the general shape of the socket 21 is substantially cylindrical or frustoconical, a rigid extrusion may not easily wrap around the socket, but will better fit along the axial orientation of the socket 21. The surrounding flexible base can then unfold and fit to the outer surface 26 of the socket 21.

In this configuration, to assemble the tibial cuff locator 340 on the socket 21, the wearer is asked to extend his or her leg and the knee orthosis 30 is positioned and the femoral cuff is fastened to the thigh of the wearer. The wearer then bends his or her leg at the knee while the knee orthosis 30 stays extended. The locator 340 is inserted into the recess 331C. However, in some embodiments, the locator 340 may be pre-inserted in the recess 331C prior to the wearer bending his or her knee. Then, a film covering an adhesive on the base 342 of the locator 340 is removed. This step may differ should another fastening mean is used. The locator 340 is still in the recess 331C. The wearer extends his or her leg until the socket 21 contacts the knee orthosis 330 to stick the locator 340 on the socket 21. The tibial cuff 331 may be secured to the socket 21 via straps. The knee orthosis 30 is removed to finish properly securing the locator 340 to the socket 21. Pressure may be applied to the base 342 of the locator 340 to ensure proper adhesion. The wearer may be asked to wait until the adhesive is properly cured.

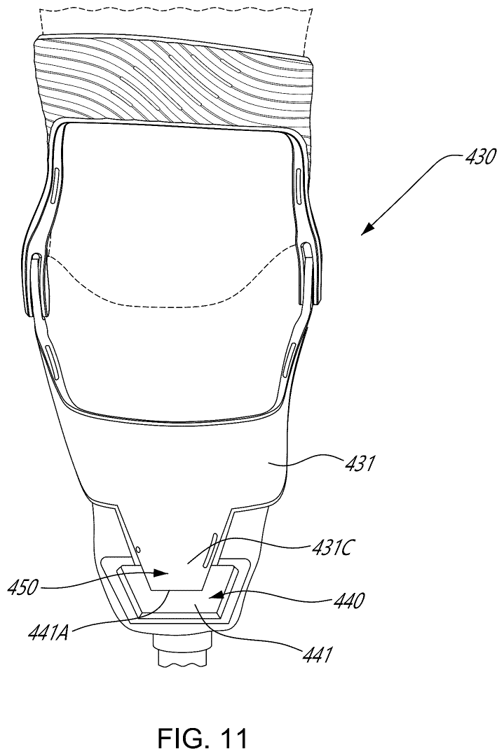

Referring to FIG. 11, another embodiment of a locator and knee orthosis are shown at 440 and 430, respectively. In this configuration, the locating member 450 of the tibial cuff 431 is a protrusion 431C defined by the tibial cuff 431 and the locator 440 is a shelf 441 that defines a cavity 441A sized to receive the protrusion 431A. The shelf 441 may be secured to the socket 21 or being an integral part of the socket 21. The cavity 441A is bounded by two lateral walls and a bottom wall. These walls may all be in contact with corresponding walls of the protrusion 431C to limit lateral and longitudinal movements of the socket 21 relative to the tibial cuff 431. The walls may be angled to guide the protrusion 431C into the cavity 441A.

In all configurations of the tibial cuff locator 40, 140, 240, 340, 440 and locating member 50, 150, 250, 350, 450 it may be said that the tibial cuff locator and the locating member each defines an abutment face. The abutment face of the locating member may be oriented away from the femoral cuff (i.e., facing downward). The abutment face on the locating member is configured to abut the tibial cuff locator, which may be defined by the leg prosthesis 20. The abutment face and the tibial cuff locator are thus adapted to abut one another to limit movements of the tibial cuff relative to the leg prosthesis in at least one direction. This direction may be parallel to a longitudinal axis of a leg of a wearer. Put differently, the locating member 50 may be a tab, a protrusion, and so on while the locator may be simply a flat wall projecting from the socket 21 to define the abutment face. In certain embodiments, the locator may be integrated to the leg prosthesis 20, for example it may be a part of the socket 21. In an alternate embodiment, the locator may be an aperture extending through the socket 21 while the locating member may be a pin protruding from the tibial cuff towards the socket and receivable within the hole.

It is noted that various connections are set forth between elements in the preceding description and in the drawings. It is noted that these connections are general and, unless specified otherwise, may be direct or indirect and that this specification is not intended to be limiting in this respect. A coupling between two or more entities may refer to a direct connection or an indirect connection. An indirect connection may incorporate one or more intervening entities. The term “connected” or “coupled to” may therefore include both direct coupling (in which two elements that are coupled to each other contact each other) and indirect coupling (in which at least one additional element is located between the two elements).

It is further noted that various method or process steps for embodiments of the present disclosure are described in the preceding description and drawings. The description may present the method and/or process steps as a particular sequence. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the description should not be construed as a limitation.

Furthermore, no element, component, or method step in the present disclosure is intended to be dedicated to the public regardless of whether the element, component, or method step is explicitly recited in the claims. As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

While various aspects of the present disclosure have been disclosed, it will be apparent to those of ordinary skill in the art that many more embodiments and implementations are possible within the scope of the present disclosure. For example, the present disclosure as described herein includes several aspects and embodiments that include particular features. Although these particular features may be described individually, it is within the scope of the present disclosure that some or all of these features may be combined with any one of the aspects and remain within the scope of the present disclosure. References to “various embodiments,” “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. The use of the indefinite article “a” as used herein with reference to a particular element is intended to encompass “one or more” such elements, and similarly the use of the definite article “the” in reference to a particular element is not intended to exclude the possibility that multiple of such elements may be present.

The embodiments described in this document provide non-limiting examples of possible implementations of the present technology. Upon review of the present disclosure, a person of ordinary skill in the art will recognize that changes may be made to the embodiments described herein without departing from the scope of the present technology. Yet further modifications could be implemented by a person of ordinary skill in the art in view of the present disclosure, which modifications would be within the scope of the present technology.

1. A knee orthosis for a trans-tibial amputee having a leg prosthesis including a socket for receiving a stump, the knee orthosis comprising:
a femoral cuff configured for engaging an upper leg portion of the trans-tibial amputee;
a tibial cuff configured for engaging the socket of the leg prosthesis, the tibial cuff having an inner face facing the socket and an outer face opposed to the inner face, the tibial cuff defining a locating member;
a hinge pivotally connecting the femoral cuff to the tibial cuff; and
a tibial cuff locator including a body protruding from a base, the base of the tibial cuff locator having a prosthesis engaging surface adapted to be mounted on the leg prosthesis, the body of the tibial cuff locator extending outwardly from the base along a locator axis, the body of the tibial cuff locator being matingly engaged with the locating member of the tibial cuff, wherein translation of the tibial cuff relative to the tibial cuff locator in one or more directions transverse to the locator axis is mitigated with the tibial cuff locator matingly engaged with the locating member of the tibial cuff.

2. The knee orthosis of claim 1, wherein the locator axis is transverse to the prosthesis engaging surface of the base of the tibial cuff locator.

3. The knee orthosis of claim 1, wherein the tibial cuff locator is adapted to be mounted on the leg prosthesis such that the tibial cuff is engageable to the socket in a single position of the tibial cuff relative to the socket.

4. The knee orthosis of claim 1, wherein the tibial cuff locator and the locating member of the tibial cuff have at least partially complementary geometries.

5. The knee orthosis of claim 1, wherein the body of the tibial cuff locator has a substantially circular cross-section transverse to the locator axis.

6. The knee orthosis of claim 1, wherein the tibial cuff locator defines a central hole extending along the locator axis and through the tibial cuff locator, the central hole sized to receive a marking device for imprinting a mark on the socket with the marking device received through the central hole.

7. The knee orthosis of claim 1, wherein the tibial cuff locator is configured to remain secured to the leg prosthesis to ensure that a position of the knee orthosis relative to the leg prosthesis is constant between successive installations of the knee orthosis.

8. The knee orthosis of claim 1, wherein the tibial cuff locator and the tibial cuff define, when mated together, a load path extending from the tibial cuff to the socket through the tibial cuff locator.

9. The knee orthosis of claim 1, wherein the locating member includes a recess defined by the tibial cuff, the body received within the recess, a shape of the body corresponding to a shape of the recess.

10. The knee orthosis of claim 1, wherein the base has a length and a width transverse to the length, the length being parallel to a socket central axis of the socket, the length greater than the width.

11. The knee orthosis of claim 9, wherein the recess is an aperture extending fully through the tibial cuff.

12. The knee orthosis of claim 1, wherein the tibial cuff locator is located on an antero-lateral side of the knee orthosis.

13. The knee orthosis of claim 1, wherein the tibial cuff locator and the locating member of the tibial cuff are matingly engaged to prevent relative rotation between the tibial cuff locator and the tibial cuff about the locator axis.

14. The knee orthosis of claim 1, wherein the body of the tibial cuff locator has a non-circular cross-sectional area taken on a plane normal to the locator axis.

15. The knee orthosis of claim 1, wherein a locking member removably fastens the knee orthosis and the tibial cuff locator together.

16. A knee orthosis for a trans-tibial amputee having a leg prosthesis including a socket for receiving a stump, the knee orthosis comprising:
a femoral cuff configured for engaging an upper leg portion of the trans-tibial amputee;
a tibial cuff configured for engaging the socket of the leg prosthesis, the tibial cuff having an inner face facing the socket and an outer face opposed to the inner face, the tibial cuff defining a locating member, the locating member defining an abutment face configured to abut a tibial cuff locator, the abutment face being transverse to the outer face of the tibial cuff, the abutment face and the tibial cuff locator adapted to abut one another to limit relative sliding movement between the tibial cuff and the leg prosthesis in one or more directions; and
a hinge pivotally connecting the femoral cuff to the tibial cuff.

17. The knee orthosis of claim 16, wherein the one or more directions includes a direction that is transverse to the tibial cuff locator and/or a direction that is parallel to a longitudinal axis of a leg of a wearer.

18. The knee orthosis of claim 16, wherein the tibial cuff locator is circular.

19. The knee orthosis of claim 16, wherein the locating member is a recess defined by the tibial cuff, the tibial cuff locator received in the recess.

20. The knee orthosis of claim 16, wherein a body of the tibial cuff locator has a non-circular cross-sectional area taken on a plane normal to a locator axis of the body of the tibial cuff locator.

21. A knee orthosis comprising:
a femoral cuff pivotally connected to a tibial cuff, the femoral cuff configured for engaging an upper leg portion of a wearer of the knee orthosis and the tibial cuff configured for engaging a lower leg portion of the wearer, the tibial cuff having an inner face and an outer face opposed to the inner face, the tibial cuff defining a locating member; and
a tibial cuff locator including a body protruding from a base, the base of the tibial cuff locator adapted to be mounted on the lower leg portion, the body of the tibial cuff locator extending outwardly from the base along a locator axis, the body of the tibial cuff locator being matingly engaged with the locating member of the tibial cuff, wherein translation of the tibial cuff relative to the tibial cuff locator in one or more directions transverse to the locator axis is mitigated with the tibial cuff locator matingly engaged with the locating member of the tibial cuff.
